(19) United States
(12) Patent Application Publication
(10) Pub. No.: US 2025/0160955 A1
(43) Pub. Date: May 22, 2025

(54) SYSTEMS AND METHODS FOR PREVENTING LASING INSIDE AN ENDOSCOPE

(71) Applicant: LUMENIS LTD., Yokneam (IL)

(72) Inventor: Vitaly Rondel, Hadera (IL)

(73) Assignee: LUMENIS LTD., Yokneam (IL)

(21) Appl. No.: 18/898,788

(22) Filed: Sep. 27, 2024

Related U.S. Application Data
(60) Provisional application No. 63/586,289, filed on Sep. 28, 2023.

(51) Int. Cl. A61B 18/26; A61B 18/00; A61B 18/20

(52) U.S. Cl. CPC A61B 18/26; A61B 2018/00511; A61B 2018/00672; A61B 2018/00678; A61B 2018/00779; A61B 2018/00982; A61B 2018/2035

(57) ABSTRACT

Described herein are laser emitting medical devices and techniques implementable by laser emitting medical devices to determine when an optical fiber is disposed within an endoscope. The devices and techniques to determine the location of the endoscope, are based on light received by the optical fiber from scope illuminating lights located on the distal end of the endoscope. An activation device enables lasing, and a computing system interrupts the lasing procedure if the optical fiber enters the endoscope.

### CROSS-REFERENCE TO RELATED APPLICATIONS

This application claims the benefit of U.S. Provisional Patent Application Ser. No. 63/586,289 filed on Sep. 28, 2024, the disclosures of which are incorporated herein by reference.

### TECHNICAL FIELD

The present disclosure generally relates to endoscopes and surgical laser systems. Particularly, but not exclusively, the disclosure relates to preventing lasing when an optical fiber coupled to the surgical laser system is inside of the endoscope.

### BACKGROUND

Endoscopes (e.g., ureteroscopes, hysteroscopes, etc.) are used in a variety of procedures. Among several of the procedures, a surgical laser system is employed to direct laser energy through an optical fiber disposed in an endoscope to specific areas of the body (e.g., endoscopic laser lithotripsy, endoscopic laser ablation, endoscopic laser coagulation, hysteroscopy etc.). During such procedures, the optical fiber is inserted into a working channel of the endoscope. The optical fiber is advanced until the distal most end of the optical fiber extends out of the distal end of the endoscope. At which point, the physician can activate the surgical laser system (e.g., via a foot pedal, or the like) to cause laser energy to be generated and directed towards a target via the optical fiber. If unintentionally, the distal end of the optical fiber is pulled back into the working channel of the endoscope while lasing, there are several potential safety issues that could arise. As such, where the distal end of the optical fiber is pulled into the working channel of the endoscope, lasing should immediately stop. Often human reaction may not be fast enough to stop lasing in a timely manner and/or the physician may not even be aware that the distal end of the optical fiber was pulled back into the endoscope. Current medical devices do not have a method for the lasing system to detect when the distal end of the optical fiber is within the endoscope. Thus, there is a need for such systems.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides an endoscopic device for use in conjunction with a surgical laser system. In general, endoscopes include at least one working channel, a camera element, and a light source for illumination. As described in greater detail below, endoscopes are often disposed in a human lumen (e.g., urinary tracts, etc.) and the light source and camera element are used to provide visual guidance for an operator to find a target (e.g., renal calculi, or the like) with the endoscope. Once the target is identified, tools (e.g., an optical fiber, etc.) can be advanced through the working channel to interact with and/or treat the target. In the example of an optical fiber, when the distal end of the optical fiber extends out of the working channel of the endoscope, the optical fiber is exposed to light from the illumination source. Likewise, when the distal end of the optical fiber is within the working channel of the endoscope, it is in darkness. The present disclosure provides a surgical lasing system configured to determine if the distal end of the optical fiber is extended out of the working channel of the endoscope or disposed within the working channel of the endoscope using a light detection system incorporated inside the lasing system.

Some embodiments of the disclosure can be implemented as a computer implemented method. The method can comprise a computer implemented method for a surgical laser console, comprising: receiving, at a processor from a light detector, an electrical signal comprising an indication of a power of light received at a distal end of an optical fiber, wherein the light corresponds to light generated by one or more illumination lights of an endoscope.

In some embodiments, the computer implemented method may further comprise determining, by the processor from the electrical signal, whether the light is greater than or equal to a threshold level. Sending a control signal to a laser source of the surgical laser console responsive to a determination that the light is not greater than or equal to the threshold level, wherein the control signal is configured to cause the laser source to stop generating laser light.

In some embodiments, the computer implemented method may further comprise receiving, at the processor from the light detector, a second electrical signal comprising an indication of the power of light received at the distal end of the optical fiber, wherein the light corresponds to light generated by the one or more illumination lights. Determining, by the processor from the second electrical signal, whether the light is greater than or equal to the threshold level, and sending a second control signal to the laser source responsive to a determination that the light is greater than or equal to the threshold level, wherein the second control signal is configured to cause the laser source to resume generating laser light.

In some embodiments of the computer implemented method, the control signal configured to cause the laser source to stop generating laser light is configured to disable the laser source.

In some embodiments of the computer implemented method, the control signal configured to cause the laser source to stop generating laser light is further configured to interrupt an activation signal from an activation device coupled to the laser console.

In some embodiments, the computer implemented method may further comprise determining, by the processor, whether the activation signal is generated by the activation device, and sending the second control signal to the laser source of the surgical laser console responsive to the determination that the light is greater than or equal to the threshold level and the determination that the activation signal is generated by the activation device.

In some embodiments of the computer implemented method, the activation device is a foot pedal.

In some embodiments, the computer implemented method further comprises determining, by the processor, whether the laser source is generating laser light and sending the control signal to the laser source of the surgical laser console responsive to the determination that the light is not greater than or equal to the threshold level and the determination that the laser source is generating laser light.

In some embodiments of the computer implemented method, the optical fiber is arranged to be inserted through a working channel of the endoscope.

In some embodiments of the computer implemented method, the optical fiber is slideably disposed within the working channel of the endoscope.

In some embodiments of the computer implemented method, the one or more illuminating lights are disposed on the distal end of the endoscope.

In some embodiments of the computer implemented method, the optical fiber is coupled to a laser console comprising the processor.

In some embodiments, a surgical laser console comprises a controller configured to implement any of the previously described methods. The console may further comprise the light detector, a laser source arranged to generate the laser light, and a beam splitter arranged to direct the laser light from the laser source to the optical fiber and arranged to direct light from the one or more illuminating lights to the light detector.

In some embodiments of the surgical laser console, the laser console further comprises an optical head comprising at least a lens arranged to couple the laser light to the optical fiber.

In some embodiments of the surgical laser console, the beam splitter comprises a dichroic mirror.

Some of the embodiments of the disclosure may be implemented as a system. The system can comprise a system for a surgical laser console, the system comprising: a processor, a light detector, and memory comprising instructions, which when executed by the processor cause the processor to receive, from the light detector, an electrical signal comprising an indication of a power of light received at a distal end of an optical fiber. Wherein the light corresponds to light generated by one or more illumination lights of an endoscope.

In some embodiment, the instructions, when executed by the processor further cause the processor to determine from the electrical signal, if the power of light received is greater than or equal to a threshold level; and send, responsive to a determination that the light is not greater than or equal to the threshold level, a control signal to a laser source of the surgical laser console, wherein the control signal is configured to cause the laser source to stop generating laser light.

In some embodiments, the instructions, when executed by the processor further cause the processor to receive from the light detector, a second electrical signal comprising an indication of the power of light received at the distal end of the optical fiber, wherein the light corresponds to light generated by the one or more illumination lights. Determine from the second electrical signal, if the power of light received is greater than or equal to a threshold level, and send, responsive to a determination that the light is greater than or equal to the threshold level, a second control signal to the laser source of the surgical laser console, wherein the second control signal is configured to cause the laser source to resume generating laser light.

In some embodiments, the control signal configured to cause the laser source to stop generating laser light, is configured to disable the laser source.

In some embodiments, the control signal configured to cause the laser source to stop generating laser light is further configured to interrupt an activation signal from an activation device coupled to the surgical laser console.

In some embodiments, the surgical laser console further comprises an activation device, wherein the instructions, when executed by the processor further cause the processor to determine whether an activation signal is generated by the activation device; and send the second control signal to the laser source of the surgical laser console responsive to the determination that the light is greater than or equal to the threshold level and the determination that the activation signal is generated by the activation device.

In some embodiments, the one or more illumination lights are disposed on the distal end of an endoscope.

In some embodiments, the optical fiber is arranged to be slideably inserted into a working channel of the endoscope.

In some embodiments, the optical fiber is coupled to a laser console comprising the processor.

In some embodiments, the laser console comprises a lasing system comprising the laser source arranged to generate laser light, and a beam splitter arranged to direct the laser light from the laser source to the optical fiber and arranged to direct light from the one or more illuminating lights to the light detector.

Some embodiments of the disclosure may be implemented as a computer-readable memory storage device (CRM), the CRM can comprise a computer-readable memory storage device comprising instructions executable by a processor of a surgical laser console, which when executed, cause the processor to receive, from a light detector, an electrical signal comprising an indication of a power of light received at a distal end of an optical fiber. The light corresponds to light generated by one or more illumination lights of an endoscope.

In some embodiments of the computer-readable memory storage device, the instructions when executed by the processor further cause the processor to determine from the electrical signal, if the power of light received is greater than or equal to a threshold level; and send, responsive to a determination that the light is not greater than or equal to the threshold level, a control signal to a laser source of the surgical laser console, wherein the control signal is configured to cause the laser source to stop generating laser light.

In some embodiments, the instructions when executed by the processor further cause the processor to receive from the light detector, a second electrical signal comprising an indication of the power of light received at the distal end of the optical fiber, wherein the light corresponds to light generated by the one or more illumination lights. Determine from the second electrical signal, if the power of light received is greater than or equal to a threshold level, responsive to a determination that the light is greater than or equal to the threshold level, a second control signal to the laser source of the surgical laser console, wherein the second control signal is configured to cause the laser source to resume generating laser light.

In some embodiments, the control signal configured to cause the laser source to stop generating laser light is further configured to interrupt an activation signal from an activation device coupled to the surgical laser console.

In some embodiments, the instructions when executed by the processor further cause the processor to Determine whether the activation signal is generated by the activation device; and send the second control signal to the laser source of the surgical laser console responsive to the determination that the light is greater than or equal to the threshold level and the determination that the activation signal is generated by the activation device.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates a medical system including an endoscope and a laser console.

FIG. 1B illustrates an example laser console.

FIG. 2A illustrates an example endoscope.

FIG. 2B illustrates an example endoscope with the optical fiber extending out of the working channel of the endoscope.

FIG. 3 illustrates a method to enable or disable lasing.

FIG. 4 illustrates graph indicating the power of light detected by the light detection system at different times.

FIG. 5 illustrates a computer-readable storage medium in accordance with one embodiment.

FIG. 6 illustrates a diagrammatic representation of a machine in the form of a computer system within which a set of instructions may be executed for causing the machine to perform any one or more of the methodologies discussed herein, according to an example embodiment.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

As noted above, medical lasers (or surgical laser systems) are used in a variety of endoscopic procedures where laser light is directed to a target through an optical fiber. One such procedure, to address renal calculi (e.g., kidney stones) is ureteral endoscopy, or lithotripsy. In a lithotripsy procedure, an endoscopic probe, with a camera and/or other sensors, is inserted into the patient's urinary tract to locate the calculi for removal. An optical fiber is inserted through a working channel of the endoscopic probe and laser energy can be directed towards the calculi via the optical fiber to disintegrate the calculi as they are found via the camera.

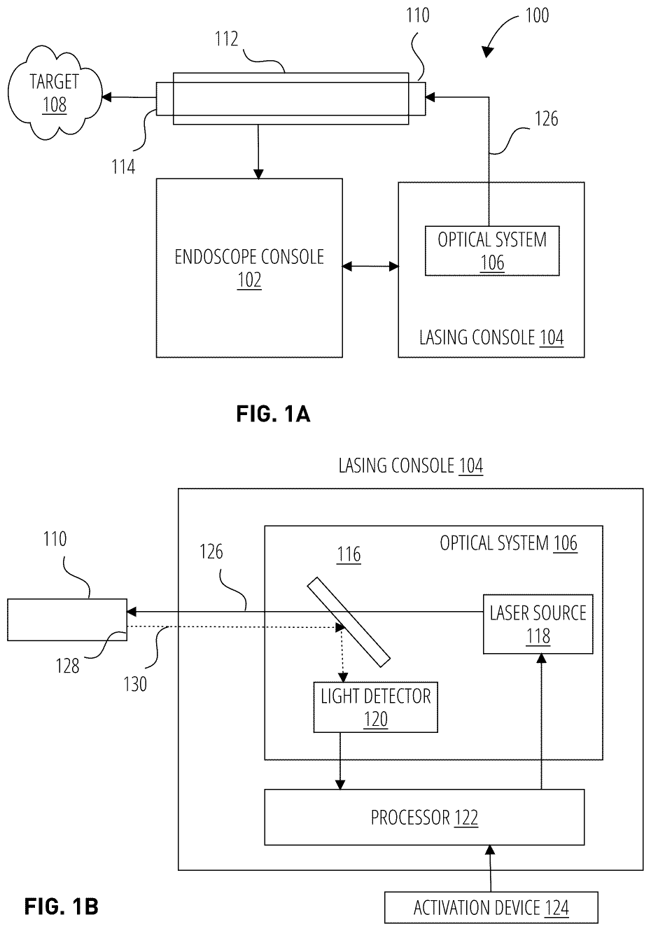

FIG. 1A show an exemplary lithotripsy system 100 configured to prevent lasing when a distal end of an optical fiber is within the endoscope, in accordance with examples of the present disclosure. Lithotripsy system 100 can comprise an endoscope console 102 and a lasing console 104. The endoscope console 102 and lasing console 104 can be coupled to the endoscope 112 and the optical fiber 110, respectively. As noted above, the endoscope 112 can include one or more working channels in which the optical fiber 110 can be inserted.

Lasing console 104 includes optical system 106, which in general includes one or more laser light sources and various optics arranged to generate a laser beam. Typically, optical system 106 will include at least two light sources; one arranged to generate an aiming beam to identify a target 108 and ensure that the optical fiber 110 is pointed at the target 108, and a second to generate a treatment beam to therapeutically treat (e.g., ablate, dust, etc.) the target 108. Such laser sources can include, but are not limited to, solid-state lasers, gas lasers, diode lasers, and fiber lasers. Further, optical system 106 includes optics to shape and/or couple the generated laser beam(s) to optical fiber 110.

The various optics within optical system 106 can include, but are not limited to, one or more polarizers, beam splitters, beam combiners, light detector, wavelength division multiplexers, collimators, circulators, and/or lenses. The laser light sources of optical system 106 can comprise a Thulium fiber laser, a Holmium laser, or other types of laser light sources.

Additionally, although endoscope console 102 and lasing console 104 are depicted as separate consoles in FIG. 1A, it is to be appreciated that an embodiment could be implemented where endoscope console 102 and lasing console 104 are combined into a single console, for example, sharing many computing components (e.g., processor, memory, display, controls, etc.). However, for purposes of clarity, the disclosure describes examples where endoscope console 102 and lasing console 104 are separate.

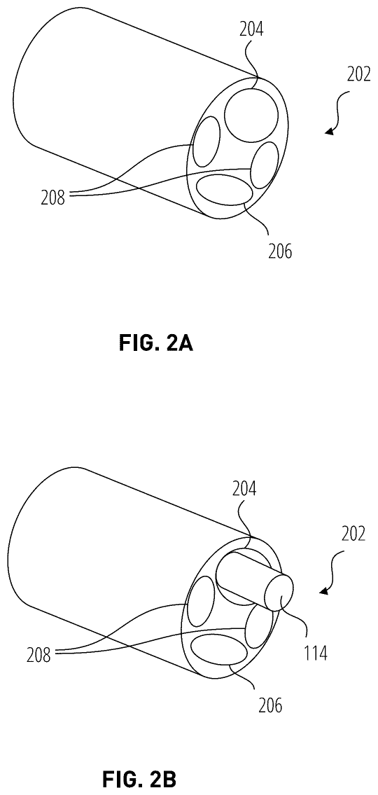

As introduced above, endoscope 112 includes a camera and lights (e.g., see FIG. 2A and FIG. 2B) with which an image of a target 108 (e.g., stone, tissue, etc.) can be acquired. In some embodiments, the target 108 may be a tissue, a stone, a tumor, a cyst, and the like, within a subject, which is to be treated, ablated, or destroyed. In some embodiments, the subject may be a human being or an animal. Once the target is acquired via the endoscope, the distal end 114 of the optical fiber 110 is advanced out of the working channel (e.g., see FIG. 2A and FIG. 2B) of the endoscope 112 and lasing can be activated to ablate, dust, or otherwise treat the target 108.

However, during a procedure, the distal end 114 of the optical fiber 110 may be inadvertently pulled back into the working channel, at which point it is unsafe to continue lasing. The present disclosure provides lasing console 104 configured to prevent lasing when the distal end 114 is within the endoscope 112.

FIG. 1B shows an example lasing console 104. The lasing console 104 may comprise an optical system 106 which includes a laser source 118, light detector 120, processor 122, and a dichroic mirror 116. As described above, the laser source 118 can be arranged to generate laser light via several different lasing mechanisms, such as, for example, using a Holmium lasing medium, using a Thulium lasing medium, or the like. Laser light may be directed to the optical fiber 110. The laser source 118 is optically coupled to the optical fiber 110. Further, the laser source 118 can be coupled to an activation device 124, to allow a user to enable lasing. In some embodiments the activation device 124 may be a pedal, button, or a foot switch. The laser source 118 is additionally coupled to processor 122. With some embodiments, the activation device 124 is coupled to laser source 118 through the processor 122 (e.g., as depicted in this figure). During operation, a user can activate the laser source 118 via the activation device 124 to cause laser source 118 to generate laser light 126, which is propagated down optical fiber 110 and can be directed towards target 108.

Additionally, during operation, the processor 122 can receive signals from light detector 120 to determine whether the distal end 114 of optical fiber 110 is outside or inside the endoscope 112 and can prevent activation of the laser source 118 (and therefore generation of laser light 126) based on such a determination.

To that end, optical system 106 includes dichroic mirror 116 and light detector 120. It will be appreciated that optical fiber 110 provides for propagation of laser light 126 from the proximal end 128 of the optical fiber 110 to the distal end 114 of the optical fiber 110. Likewise, optical fiber 110 provides for propagation of light from the distal end 114 of optical fiber 110 to the proximal end 128 of optical fiber 110. As such, ambient light 130 from the environment in which the endoscope 112 and target 108 are disposed will be propagated back up the optical fiber towards the lasing console 104. As a specific example, light from lights of the endoscope will be transmitted from the distal end 114 to the proximal end 128 of the optical fiber 110.

Lasing console 104 includes dichroic mirror 116 positioned in the optical path between optical fiber 110 and laser source 118 and configured to pass substantially all of light having a wavelength within a first wavelength range such that laser light 126 is optically conveyed from laser source 118 to the proximal end 128 of optical fiber 110. Further, dichroic mirror 116 is positioned and configured to reflect light having a wavelength within a second wavelength range such that substantially all ambient light 130 is reflected towards light detector 120. In general, light detector 120 is arranged to measure and/or generate an electrical signal indicative of the intensity of light incident on the light detector 120. In the context of the present disclosure, light detector 120 is configured to generate electrical signals comprising indications of the intensity of ambient light 130. These electrical signals can be communicated to a computing device (e.g., processor 122 and the like) to determine the intensity or ambient light 130. It is noted that although the term ambient light is used, the environment may be void of actual ambient light and may only have light from lasing and light from the endoscope lights (or environmental light). However, light not from lasing (e.g., light from endoscope lights, or the like) is referred to herein as ambient light for convenience.

As stated above, during lasing procedures, it may be important to stop lasing when the distal end 114 of the optical fiber 110 is inside of the endoscope 112. The processor 122 can be configured to stop lasing (e.g., interrupt laser source 118, or the like) if the intensity of ambient light 130 decreases below a first threshold level. The processor 122 may also be configured to enable lasing if the intensity of ambient light 130 increases above a second threshold level, which can be the same or different than the first threshold level. Accordingly, activation device 124 enables laser source 118 to generate laser light 126, while the processor 122 may interrupt the generation of laser light 126 if the intensity of ambient light 130 decreases below the threshold and may only allow reactivation or regeneration of laser light 126 if both activation device 124 is activated and the intensity of ambient light 130 increases above the threshold.

FIG. 2A illustrates an example of a distal end 202 of the endoscope 112. As depicted in this figure, the endoscope 112 may comprise a working channel 204, through which the optical fiber 110 may be inserted. The distal end 202 of the endoscope 202 also can include Camera 206 and lights 208, configured to illuminate the target and provide imaging information. Although distal end 202 is shown with two lights 208, it can be provided with one or more (e.g., two, three, etc.) lights 208.

As described above, during operation, the distal end 114 of optical fiber 110 is advanced down the working channel 204 and advanced out of the distal end 202 of the endoscope 112. However, prior to being advanced out of the distal end 202 of the endoscope 112 or if inadvertently pulled back into the working channel 204 of the endoscope 112, the distal end 114 of the optical fiber 110 is not exposed to the light from lights 208. As such, ambient light 130 is not propagated up the optical fiber to the light detector 120 as described above. In such instances, the light detector 120 will measure and/or detect ambient light 130 below a threshold level, which can trigger processor 122 to disable or prevent lasing by laser source 118 as described above.

FIG. 2B illustrates the distal end of an endoscope 202, when the distal end 114 of the optical fiber 110 is extending out of the working channel 204. When the distal end 114 of the optical fiber 110 is outside of the working channel 204, it is exposed to light from the lights 208. As such, this light will propagate up optical fiber 110 as ambient light 130, be reflected by dichroic mirror 116 and be incident on light detector 120. Light detector 120 can measure the intensity of the ambient light 130 and generate an electrical signal indicative of the measured intensity, which can trigger processor 122 to allow lasing by laser source 118 as described above.

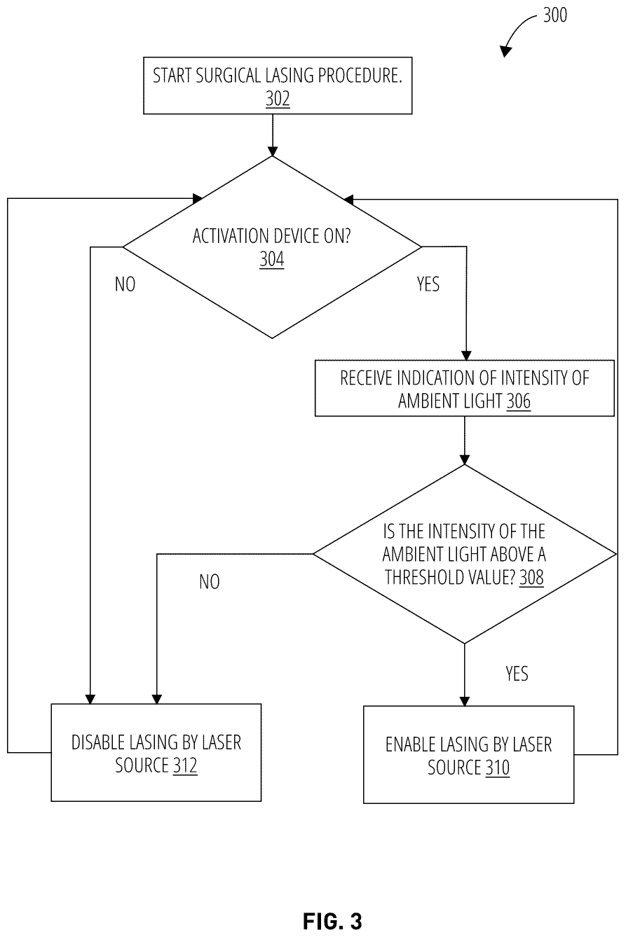

FIG. 3 illustrates a method 300 for a lasing console to enable or disable lasing to prevent lasing within the endoscope as outlined herein. For example, method 300 can be implemented by lithotripsy system 100 to prevent lasing of laser source 118 when the distal end 114 of optical fiber 110 is within the endoscope 112. However, method 300 could be implemented by another system different than lithotripsy system 100. Examples are not limited by this context. Method 300 can begin at block 302. At block 302 “start surgical lasing procedure” a physician begins a procedure. For example, endoscope 112 can be inserted into a treatment area (e.g., an environment to be treated as part of a lithotripsy procedure, a patients urinary or biliary tract, or the like). In some embodiments, the distal end 202 of the endoscope 112 may be positioned in the environment including a target 108. With some embodiments, method 300 may not include block 302, in which case, operations by the physician and/or operations for placing the endoscope etc., are not included as part of method 300.

Continuing to block 304 “is activation device on?” a determination is made as to whether the activation device is on. For example, processor 122 can determine whether activation device 124 is “on” (e.g., activated). With some embodiments, processor 122 can execute instructions (e.g., stored in a computer-readable memory storage device, or the like) to determine whether activation device 124 is activated. From decision block 304, method 300 can continue to either block 306 or block 312. Method 300 can continue from decision block 304 to block 306 based on a determination at decision block 304 that the activation device is on while method 300 can continue from decision block 304 to block 312 based on a determination at decision block 304 that the activation device is not on.

At block 306 “receive indication of intensity of ambient light” an indication of an intensity of ambient light can be received. For example, processor 122 can receive an indication of an intensity of ambient light 130 from light detector 120.

Continuing to decision block 308 “is the intensity of the ambient light above a threshold value?” a determination is made as to whether the intensity of the ambient light is above a threshold value. For example, processor 122 can determine whether the intensity of the ambient light 130 is above a threshold value. From decision block 308, method 300 can continue to either block 312 or block 310. Method 300 can continue from decision block 308 to block 310 based on a determination at decision block 308 that the intensity of the ambient light is above (e.g., greater than or equal to, or the like) the threshold level while method 300 can continue from decision block 308 to block 312 based a determination at decision block 308 that the intensity of the ambient light is not above (e.g., less than or equal to, or the like) the threshold level.

It is to be appreciated, that increased levels of ambient light 130 correlate with the distal end 114 of the optical fiber 110 extending from the distal end 202 of the endoscope 112. When the distal end 114 of the optical fiber 110 is a certain distance outside of the endoscope 112, it is safe to enable lasing. When the optical fiber is within the endoscope 112, it will not be exposed to the scope illuminating lights 208, and the light detector 120 won't detect light, or will detect light below the threshold level. When the level of light detected by the light detector 120 is below the threshold, lasing should be disabled. Said differently, lasing should be enabled when both (1) the activation device is on and (2) the intensity of ambient light 130 is above the threshold level.

At block 310 “enable lasing by the laser source” lasing by the laser source is enabled. For example, processor 122 can send a control signal to laser source 118 to cause laser source 118 to generate laser light 126. That is, when both (1) the activation device is on and (2) the intensity of ambient light 130 is above the threshold level as determined at decision block 304 and decision block 308, processor 122 can send a control signal to laser source 118 to activate laser source 118.

At block 312 “disable lasing by the laser source” lasing by the laser source is disabled. For example, processor 122 can send a control signal to laser source 118 to cause laser source 118 to stop generating laser light 126. That is, when either (1) the activation device is not on or (2) the intensity of ambient light 130 is below the threshold level as determined at decision block 304 and decision block 308, processor 122 can send a control signal to laser source 118 to disable or deactivate the laser source 118.

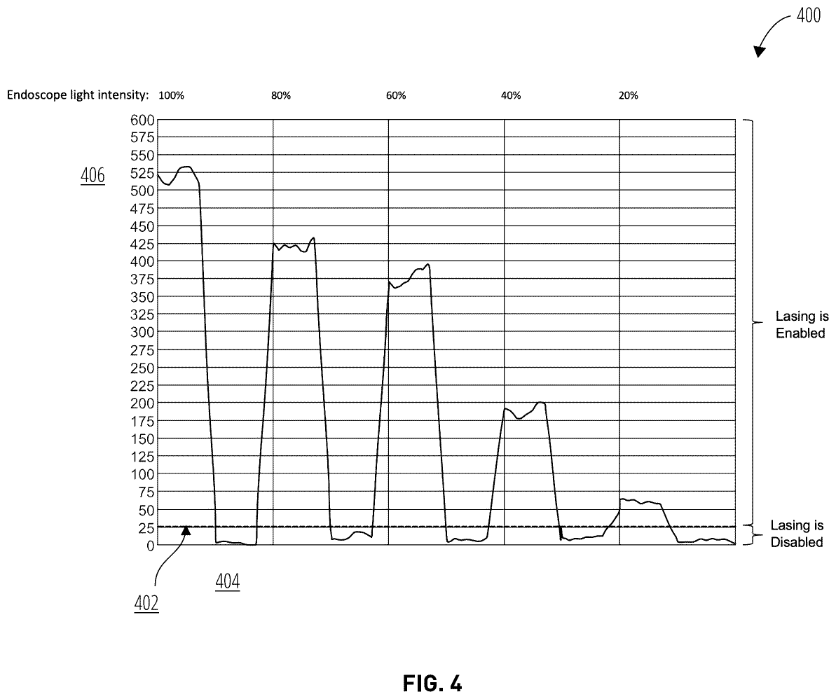

FIG. 4 illustrates a graph 400 showing the intensity of ambient light at various threshold levels. For example, the intensity of ambient light is depicted on the y axis 406. This intensity can correspond to the intensity of light received at the light detector 120 and/or as determined by processor 122. As shown in this figure, as the distal end 114 of the optical fiber 110 is pulled into the endoscope 112, the intensity of the ambient light 130 decreases. When the intensity of the ambient light 130 is below a threshold 402, lasing can be interrupted as described herein. Further, when the distal end 114 of the optical fiber 110 extends out of the endoscope 112, the intensity of the ambient light 130 detected by the light detector 120 and/or determined by processor 122 increase. When the intensity is above the threshold 402, lasing can be enabled. This figure also illustrates the change in intensity of ambient light 130 when the endoscope lights 208 are set at varying level of output power. For example, even when the endoscope illuminating lights 208 are at 20% intensity, there is still a clear difference between the intensity of ambient light 130 when the distal end 114 of the optical fiber 110 is outside the endoscope 112 and the threshold 402.

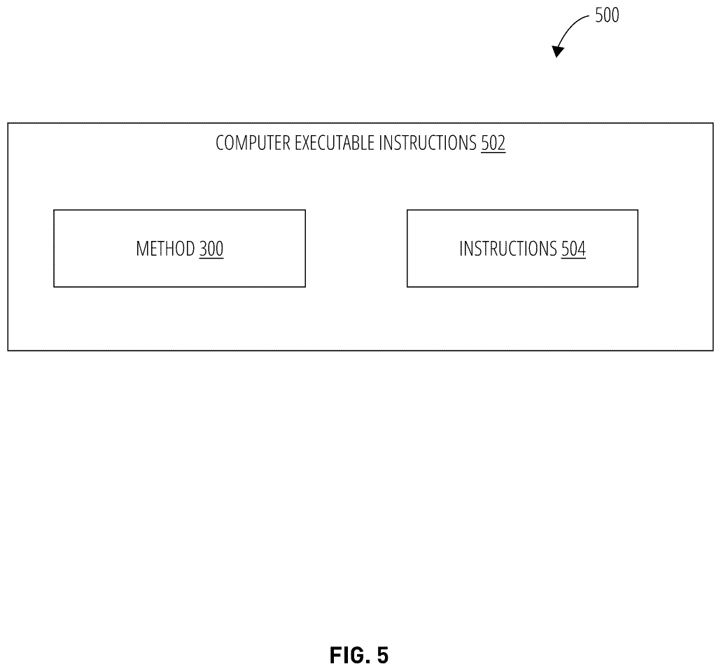

FIG. 5 illustrates computer-readable storage medium 500. Computer-readable storage medium 500 may comprise any non-transitory computer-readable storage medium or machine-readable storage medium, such as an optical, magnetic or semiconductor storage medium. In various embodiments, computer-readable storage medium 500 may comprise an article of manufacture. In some embodiments, computer-readable storage medium 500 may store computer executable instructions 502 with which circuitry can execute. For example, computer executable instructions 502 can include instructions 504 executable by a processor (e.g., processor 122, or the like) to enable or disable lasing as described herein. As another example, computer executable instructions 502 can include instructions corresponding to method 300 of FIG. 3 configured to cause a lithotripsy system 100 to implement the method 300. Examples of computer-readable storage medium 500 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 502 may include any suitable type of code, such as source code, compiled code, interpreted code, executable code, static code, dynamic code, object-oriented code, visual code, and the like.

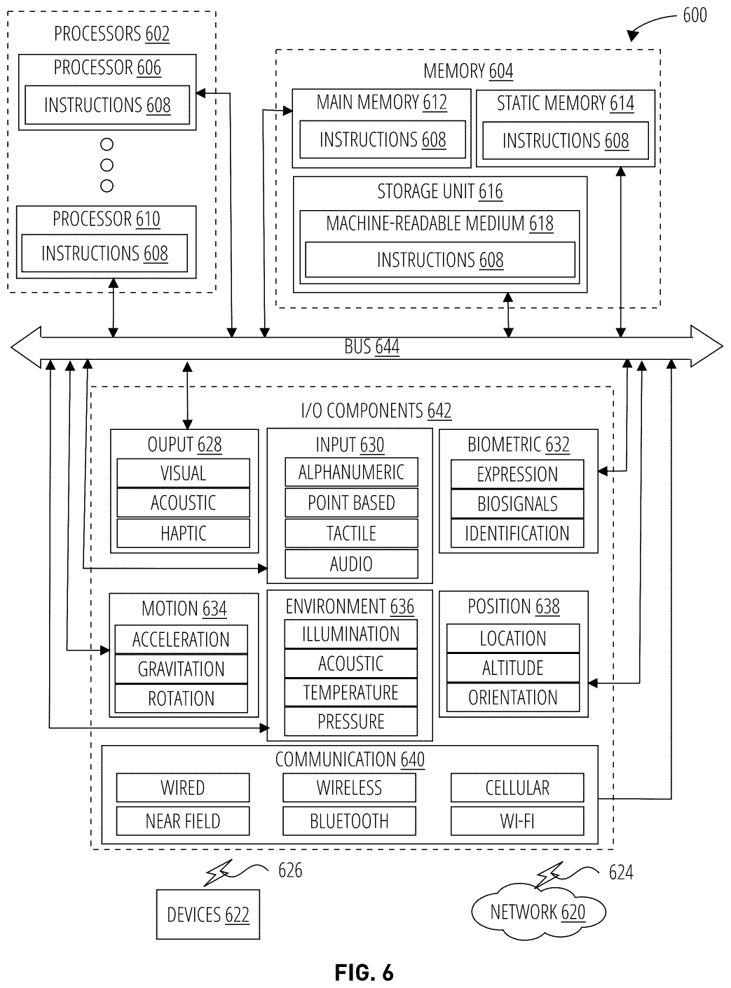

FIG. 6 illustrates a diagrammatic representation of a machine 500 in the form of a computer system within which a set of instructions may be executed for causing the machine to perform any one or more of the methodologies discussed herein. More specifically, FIG. 6 shows a diagrammatic representation of the machine 600 in the example form of a computer system, within which instructions 608 (e.g., software, a program, an application, an applet, an app, or other executable code) for causing the machine 600 to perform any one or more of the methodologies discussed herein may be executed. For example, the instructions 608 may cause the machine 600 to execute method 300, or the like. More generally, the instructions 608 may cause the machine 600 to enable or disable lasing to prevent lasing when the distal end 114 of an optical fiber 110 is within the endoscope 112.

The instructions 608 transform the general, non-programmed machine 600 into a particular machine 600 programmed to carry out the described and illustrated functions in a specific manner. In alternative embodiments, the machine 600 operates as a standalone device or may be coupled (e.g., networked) to other machines. In a networked deployment, the machine 600 may operate in the capacity of a server machine or a client machine in a server-client network environment, or as a peer machine in a peer-to-peer (or distributed) network environment. The machine 600 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 608, sequentially or otherwise, that specify actions to be taken by the machine 600. Further, while only a single machine 600 is illustrated, the term “machine” shall also be taken to include a collection of machines 200 that individually or jointly execute the instructions 608 to perform any one or more of the methodologies discussed herein.

The machine 600 may include processors 602, memory 604, and I/O components 642, which may be configured to communicate with each other such as via a bus 644. In an example embodiment, the processors 602 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 606 and a processor 610 that may execute the instructions 608. The term “processor” is intended to include multi-core processors that may comprise two or more independent processors (sometimes referred to as “cores”) that may execute instructions contemporaneously. Although FIG. 6 shows multiple processors 602, the machine 600 may include a single processor with a single core, a single processor with multiple cores (e.g., a multi-core processor), multiple processors with a single core, multiple processors with multiples cores, or any combination thereof.

The memory 604 may include a main memory 612, a static memory 614, and a storage unit 616, both accessible to the processors 602 such as via the bus 644. The main memory 604, the static memory 614, and storage unit 616 store the instructions 608 embodying any one or more of the methodologies or functions described herein. The instructions 608 may also reside, completely or partially, within the main memory 612, within the static memory 614, within machine-readable medium 618 within the storage unit 616, within at least one of the processors 602 (e.g., within the processor's cache memory), or any suitable combination thereof, during execution thereof by the machine 600.

The I/O components 642 may include a wide variety of components to receive input, provide output, produce output, transmit information, exchange information, capture measurements, and so on. The specific I/O components 642 that are included in a particular machine will depend on the type of machine. For example, portable machines such as mobile phones will likely include a touch input device or other such input mechanisms, while a headless server machine will likely not include such a touch input device. It will be appreciated that the I/O components 642 may include many other components that are not shown in FIG. 6. The I/O components 642 are grouped according to functionality merely for simplifying the following discussion and the grouping is in no way limiting. In various example embodiments, the I/O components 642 may include output components 628 and input components 630. The output components 628 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 630 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 642 may include biometric components 632, motion components 634, environmental components 636, or position components 638, among a wide array of other components. For example, the biometric components 632 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 634 may include acceleration sensor components (e.g., accelerometer), gravitation sensor components, rotation sensor components (e.g., gyroscope), and so forth. The environmental components 636 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 638 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 642 may include communication components 640 operable to couple the machine 600 to a network 620 or devices 622 via a coupling 624 and a coupling 626, respectively. For example, the communication components 640 may include a network interface component or another suitable device to interface with the network 620. In further examples, the communication components 640 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 622 may be another machine or any of a wide variety of peripheral devices (e.g., a peripheral device coupled via a USB).

Moreover, the communication components 640 may detect identifiers or include components operable to detect identifiers. For example, the communication components 640 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph®, MaxiCode, PDF417, Ultra Code, UCC RSS-2D bar code, and other optical codes), or acoustic detection components (e.g., microphones to identify tagged audio signals). In addition, a variety of information may be derived via the communication components 640, such as location via Internet Protocol (IP) geolocation, location via Wi-Fi® signal triangulation, location via detecting an NFC beacon signal that may indicate a particular location, and so forth.

The various memories (i.e., memory 604, main memory 612, static memory 614, and/or memory of the processors 602) and/or storage unit 616 may store one or more sets of instructions and data structures (e.g., software) embodying or utilized by any one or more of the methodologies or functions described herein. These instructions (e.g., the instructions 608), when executed by processors 602, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 620 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 620 or a portion of the network 620 may include a wireless or cellular network, and the coupling 624 may be a Code Division Multiple Access (CDMA) connection, a Global System for Mobile communications (GSM) connection, or another type of cellular or wireless coupling. In this example, the coupling 624 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 608 may be transmitted or received over the network 620 using a transmission medium via a network interface device (e.g., a network interface component included in the communication components 640) and utilizing any one of several well-known transfer protocols (e.g., hypertext transfer protocol (HTTP)). Similarly, the instructions 608 may be transmitted or received using a transmission medium via the coupling 626 (e.g., a peer-to-peer coupling) to the devices 622. The terms “transmission medium” and “signal medium” mean the same thing and may be used interchangeably in this disclosure. The terms “transmission medium” and “signal medium” shall be taken to include any intangible medium that can store, encoding, or carrying the instructions 608 for execution by the machine 600, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A system for a surgical laser console, the system comprising:
a processor;
a light detector; and
memory comprising instructions, which when executed by the processor cause the processor to:
receive, from the light detector, an electrical signal comprising an indication of a power of light received at a distal end of an optical fiber,
wherein the light corresponds to light generated by one or more illumination lights of an endoscope.

2. The system of claim 1, the instructions, when executed by the processor further cause the processor to:
determine from the electrical signal, if the power of light received is greater than or equal to a threshold level; and send, responsive to a determination that the light is not greater than or equal to the threshold level, a control signal to a laser source of the surgical laser console, wherein the control signal is configured to cause the laser source to stop generating laser light.

3. The system of claim 2, the instructions, when executed by the processor further cause the processor to:
receive from the light detector, a second electrical signal comprising an indication of the power of light received at the distal end of the optical fiber, wherein the light corresponds to light generated by the one or more illumination lights;
determine from the second electrical signal, if the power of light received is greater than or equal to a threshold level; and send, responsive to a determination that the light is greater than or equal to the threshold level, a second control signal to the laser source of the surgical laser console, wherein the second control signal is configured to cause the laser source to resume generating laser light.

4. The system of claim 2, wherein the control signal configured to cause the laser source to stop generating laser light, is configured to disable the laser source.

5. The system of claim 4, wherein the control signal configured to cause the laser source to stop generating laser light is further configured to interrupt an activation signal from an activation device coupled to the surgical laser console.

6. The system of claim 3, further comprising an activation device, wherein the instructions, when executed by the processor further cause the processor to:
determine whether an activation signal is generated by the activation device;
and send the second control signal to the laser source of the surgical laser console responsive to the determination that the light is greater than or equal to the threshold level and the determination that the activation signal is generated by the activation device.

7. The system of claim 4, wherein the one or more illumination lights are disposed on the distal end of an endoscope.

8. The system of claim 7, wherein the optical fiber is arranged to be slideably inserted into a working channel of the endoscope.

9. The system of claim 8, wherein the optical fiber is coupled to a laser console comprising the processor.

10. The system of claim 9, wherein the laser console comprises a lasing system comprising:
the laser source arranged to generate laser light; and
a beam splitter arranged to direct the laser light from the laser source to the optical fiber and arranged to direct light from the one or more illuminating lights to the light detector.

11. A computer-readable memory storage device comprising instructions executable by a processor of a surgical laser console, which when executed, cause the processor to:
receive, from a light detector, an electrical signal comprising an indication of a power of light received at a distal end of an optical fiber, wherein the light corresponds to light generated by one or more illumination lights of an endoscope.

12. The computer-readable memory storage device of claim 11, the instructions when executed by the processor further cause the processor to:
determine from the electrical signal, if the power of light received is greater than or equal to a threshold level; and
send, responsive to a determination that the light is not greater than or equal to the threshold level, a control signal to a laser source of the surgical laser console, wherein the control signal is configured to cause the laser source to stop generating laser light.

13. The computer-readable memory storage device of claim 12, the instructions when executed by the processor further cause the processor to:
receive from the light detector, a second electrical signal comprising an indication of the power of light received at the distal end of the optical fiber, wherein the light corresponds to light generated by the one or more illumination lights;
determine from the second electrical signal, if the power of light received is greater than or equal to a threshold level; and
send, responsive to a determination that the light is greater than or equal to the threshold level, a second control signal to the laser source of the surgical laser console, wherein the second control signal is configured to cause the laser source to resume generating laser light.

14. The computer-readable memory storage device of claim 13, wherein the control signal configured to cause the laser source to stop generating laser light is further configured to interrupt an activation signal from an activation device coupled to the surgical laser console.

15. The computer-readable memory storage device of claim 14, the instructions when executed by the processor further cause the processor to:
determine whether the activation signal is generated by the activation device; and
send the second control signal to the laser source of the surgical laser console responsive to the determination that the light is greater than or equal to the threshold level and the determination that the activation signal is generated by the activation device.

16. A computer implemented method for a surgical laser console, comprising:
receiving, at a processor from a light detector, an electrical signal comprising an indication of a power of light received at a distal end of an optical fiber, wherein the light corresponds to light generated by one or more illumination lights of an endoscope.

17. The computer implemented method of claim 16, further comprising:
determining, by the processor from the electrical signal, whether the light is greater than or equal to a threshold level; and
sending a control signal to a laser source of the surgical laser console responsive to a determination that the light is not greater than or equal to the threshold level, wherein the control signal is configured to cause the laser source to stop generating laser light.

18. The computer implemented method of claim 17, further comprising:
receiving, at the processor from the light detector, a second electrical signal comprising an indication of the power of light received at the distal end of the optical fiber, wherein the light corresponds to light generated by the one or more illumination lights;
determining, by the processor from the second electrical signal, whether the light is greater than or equal to the threshold level; and
sending a second control signal to the laser source responsive to a determination that the light is greater than or equal to the threshold level, wherein the second control signal is configured to cause the laser source to resume generating laser light.

19. The computer implemented method of claim 18, wherein the control signal configured to cause the laser source to stop generating laser light is configured to disable the laser source.

20. The computer implemented method of claim 18, wherein the control signal configured to cause the laser source to stop generating laser light is further configured to interrupt an activation signal from an activation device coupled to the laser console and wherein the method further comprises:
determining, by the processor, whether the activation signal is generated by the activation device; and
sending the second control signal to the laser source of the surgical laser console responsive to the determination that the light is greater than or equal to the threshold level and the determination that the activation signal is generated by the activation device.
